(19) United States
(12) Patent Application Publication
(10) Pub. No.: US 2026/0049047 A1
(43) Pub. Date: Feb. 19, 2026

(54) METHOD OF PRODUCING FLUOROOLEFIN

(71) Applicant: AGC INC., Tokyo (JP)

(72) Inventors: Hikaru IWASAKI, Tokyo (JP); Taku YAMADA, Tokyo (JP)

(73) Assignee: AGC INC., Tokyo (JP)

(21) Appl. No.: 19/369,114

(22) Filed: Oct. 24, 2025

Related U.S. Application Data
(63) Continuation of application No. PCT/JP2024/013506, filed on Apr. 1, 2024.

(30) Foreign Application Priority Data
Apr. 26, 2023 (JP) 2023-072720

(51) Int. Cl. C07C 17/357; B01J 21/04; C07C 21/18

(52) U.S. Cl. CPC C07C 17/357; C07C 21/18; B01J 21/04

(57) ABSTRACT

A method of producing fluoroolefin includes contacting a fluorocarbon represented by Formula (1): CX¹X²F—CX³X⁴H with a catalyst to produce a fluoroolefin represented by Formula (2): CX¹X²═CX³X⁴, and satisfies either (I) or (II). (I) The catalyst includes 65% by mass or more of α-alumina and has an amount of Lewis acid sites of from 0.005 to 0.10 mmol/g as measured by ammonia temperature-programmed desorption method. (II) A conversion rate 10 hours after contacting the fluorocarbon with the catalyst is from 7.0 to 13.0%, and a conversion retention rate, which is a ratio of the conversion rate 50 hours after contact to a conversion rate after 10 hours, is 69% or higher.

### CROSS-REFERENCE TO RELATED APPLICATIONS

This application is a Continuation of International Application No. PCT/JP2024/013506, filed Apr. 1, 2024, which claims priority to Japanese Patent Application No. 2023-072720 filed Apr. 26, 2023. Each of the above applications is hereby expressly incorporated by reference, in its entirety, into the present application.

### TECHNICAL FIELD

The present disclosure relates to a method of producing a fluoroolefin.

### BACKGROUND ART

In recent years, fluoroolefins have attracted attention as compounds with low global warming potential.

For example, Patent Document 1 describes a method of producing fluoroolefins that includes a dehydrofluorination process in which fluorocarbons are dehydrofluorinated by contacting them with a metal catalyst. The dehydrofluorination process is carried out in a gas phase in the presence of water, with the water concentration with respect to the fluorocarbon being 500 ppm, in which y-alumina is used as a catalyst. Furthermore, Non-Patent Document I describes a method for obtaining trifluoroethylene using α-alumina, y-alumina, 0-alumina or the like in the dehydrofluorination reaction of 1,1,1,2-tetrafluoroethane.

### PRIOR ART DOCUMENTS

### Patent Documents

Patent Document 1: Japanese Patent Application Laid-Open (JP-A) 2019-196347

### Non-Patent Documents

Non-Patent Document 1: Catalysis Letters 2015, Vol. 145, pp. 654-661

### SUMMARY OF INVENTION

However, as described in Patent Document 1, in a case in which y-alumina is used as a catalyst, a conversion rate tended to decrease over long periods of production. Furthermore, as described in Non-Patent Document 1, in a case in which y-alumina, 0-alumina, or a combination thereof is used as a catalyst, an initial conversion rate was high, but a conversion rate decreased over long periods of production. Furthermore, in a case in which α-alumina is used as a catalyst, the conversion rate was extremely low at 1.2%.

An object of one embodiment in the present invention is to provide a method of producing a fluoroolefin, which achieves a higher conversion rate than conventional methods and suppresses a decrease in conversion rate during long-term production.

### Solution to Problem

The present disclosure includes the following aspects.
<1> A method of producing a fluoroolefin, the method including contacting a fluorocarbon represented by the following Formula (1) with a catalyst to produce a fluoroolefin represented by the following Formula (2),
   in which the catalyst includes 65% by mass or more of α-alumina and has an amount of Lewis acid sites of from 0.005 to 0.10 mmol/g as measured by ammonia temperature-programmed desorption method.

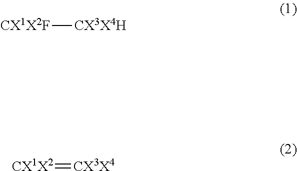

in which, in Formulas (1) and (2), X¹, X², X³ and X⁴ each independently represent a hydrogen atom or a fluorine atom, provided that at least one of X¹, X² X³ or X⁴ is a fluorine atom.
<2> The method of producing a fluoroolefin according to <1>, in which the amount of Lewis acid sites is from 0.005 to 0.05 mmol/g.
<3> The method of producing a fluoroolefin according to <1> or <2>, in which the amount of Lewis acid sites is from 0.008 to 0.05 mmol/g.
<4> The method of producing a fluoroolefin according to any one of <1> to <3>, in which an amount of fluorocarbon supplied to the catalyst is 350 kg/hr/m³ or more.
<5> The method of producing a fluoroolefin according to any one of <1> to <4>, in which the fluorocarbon is at least one selected from the group consisting of 1,1,1-trifluoroethane, 1,1,2-trifluoroethane, 1,1,2,2-tetrafluoroethane, and 1,1, 1,2-tetrafluoroethane.
<6> The method of producing a fluoroolefin according to any one of <1> to <5>, in which the fluoroolefin is at least one selected from the group consisting of 1,2-difluoroethylene, 1,1-difluoroethylene, and trifluoroethylene.

The method of producing a fluoroolefin according to any one of <1> to <6>, in which the fluorocarbon is 1,1,1,2-tetrafluoroethane, and the fluoroolefin is trifluoroethylene.
<8> The method of producing a fluoroolefin according to any one of <1> to <7>, in which the fluorocarbon and the catalyst are contacted at a temperature of from 300 to 800° C.
<9> The method of producing a fluoroolefin according to any one of <1> to <8>, in which:
   the fluorocarbon and the catalyst are contacted in the presence of an inert gas; and
   the inert gas is at least one selected from the group consisting of nitrogen, helium, argon, octafluorocyclobutane, and carbon dioxide.
<10> The method of producing a fluoroolefin according to any one of <1> to <9>, further including drying the catalyst before contacting the fluorocarbon with the catalyst.
<11> The method of producing a fluoroolefin according to any one of <1> to <10>, in which:
   the fluorocarbon and the catalyst are contacted in a gas phase in the presence of water; and
   a concentration of the water is less than 500 ppm by mass with respect to a total amount of a feed gas containing the fluorocarbon.
<12> A method of producing a fluoroolefin, the method including contacting a fluorocarbon represented by the following Formula (1) with a catalyst to produce a fluoroolefin represented by the following Formula (2),
   in which a conversion rate 10 hours after contacting the fluorocarbon with the catalyst is from 7.0 to 13%, and a conversion retention rate, which is a ratio of the conversion rate 50 hours after contact to the conversion rate after 10 hours, is 69% or higher.

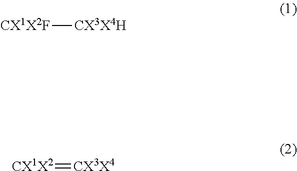

in which, in Formulas (1) and (2), X¹, X², X³ and X⁴ each independently represent a hydrogen atom or a fluorine atom, provided that at least one of X¹, X², X³ or X⁴ is a fluorine atom.

### Advantageous Effects of Invention

According to the present disclosure, it is possible to provide a method of producing a fluoroolefin, which achieves a higher conversion rate than conventional methods and suppresses a decrease in conversion rate during long-term production.

### BRIEF DESCRIPTION OF DRAWINGS

FIG. 1 is a diffraction pattern obtained by XRD measurement of the catalyst of Example 6.

### DESCRIPTION OF EMBODIMENTS

In the present disclosure, numerical ranges indicated using “to” refer to ranges that include the numerical values before and after “to” as the minimum and maximum values, respectively.

In numerical ranges described in a stepwise manner in the present disclosure, the upper limit value or lower limit value of one range may be replaced with the upper limit value or lower limit value of another stepwise-described range. Additionally, in the numerical ranges described in the present disclosure, the upper limit value or lower limit value of one range may be replaced with a value shown in the examples.

In the present disclosure, a combination of two or more preferred aspects is a more preferred aspect.

In the present disclosure, the amount of each component in a composition refers, unless otherwise specified, to the total amount of multiple substances corresponding to that component in a case in which multiple such substances are present in the composition.

In the present disclosure, “long-term production” refers to production for 50 hours by contacting a fluorocarbon with a catalyst, and may also refer to production for 50 hours or more. [Method of Producing Fluoroolefin]

A method of producing a fluoroolefin of a first embodiment in the present disclosure includes contacting a fluorocarbon represented by the following Formula (1) with a catalyst to produce a fluoroolefin represented by the following Formula (2), in which the catalyst includes 65% by mass or more of α-alumina and has an amount of Lewis acid sites of 0.005 to 0.10 mmol/g as measured by ammonia temperature-programmed desorption method.

A method of producing a fluoroolefin of a second embodiment in the present disclosure includes contacting a fluorocarbon represented by the following Formula (1) with a catalyst to produce a fluoroolefin represented by the following Formula (2), in which a conversion rate 10 hours after contacting the fluorocarbon with the catalyst is from 7.0 to 13.0%, and a conversion retention rate, which is a ratio of the conversion rate 50 hours after contact to a conversion rate after 10 hours, is 69% or higher.

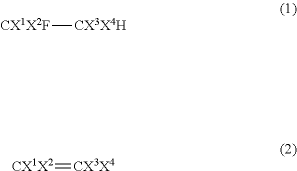

In Formulas (1) and (2), X¹, X², X³ and X⁴ each independently represent a hydrogen atom or a fluorine atom, provided that at least one of X¹, X², X³ or X⁴ is a fluorine atom.

The method of producing a fluoroolefin of the first embodiment and the method of producing a fluoroolefin of the second embodiment are collectively referred to as a fluoroolefin production method in the present disclosure.

According to the fluoroolefin production method in the present disclosure, it is possible to achieve a higher conversion rate than conventional methods, and suppress a decrease in conversion rate during long-term production. The reasons for this are not clear, but are presumed to be as follows.

In the reaction from the fluorocarbon represented by Formula (1) to the fluoroolefin represented by Formula (2), hydrogen fluoride is generated. The generated hydrogen fluoride reacts with the fluoroolefin represented by Formula (2) to revert to the fluorocarbon represented by Formula (1). Because this reaction proceeds, the conversion rate of the fluorocarbon represented by Formula (1) has traditionally been very low.

In contrast, the present inventors have found that in a case in which of a catalyst containing 65% by mass or more of α-alumina and having an amount of Lewis acid sites of 0.005 mmol/g as measured by ammonia temperature-programmed desorption method is used, the conversion rate is improved. This is considered to be because active sites that activate C—F bonds are present in an effective amount in the catalyst, thereby exhibiting reaction activity for dehydrofluorination.

Furthermore, the present inventors have found that in a case in which of a catalyst containing 65% by mass or more of α-alumina and having an amount of Lewis acid sites of 0.10 mmol/g or less as measured by ammonia temperature-programmed desorption method is used, a decrease in catalytic activity during long-term production is suppressed. This is considered to be because fluorination of the catalyst by hydrogen fluoride generated during the reaction is appropriately suppressed. It is also considered to be because coking, a phenomenon in which carbon generated by decomposition of the raw material accumulates on the catalyst, is appropriately suppressed.

On the other hand, Patent Document 1 describes a method of producing a fluoroolefin using γ-alumina. However, since γ-alumina undergoes significant changes in its crystal structure under high-temperature conditions, in a case in which γ-alumina is used as a catalyst, the conversion rate tends to decrease during long-term production.

In addition, Non-Patent Document 1 describes a method of producing trifluoroethylene using Al₂O₃-1200, which contains 100% by mass of α-alumina. According to analyses by the ammonia temperature-programmed desorption method (NH₃-TPD) or the IR spectrum of adsorbed pyridine (Py-IR). Al₂O₃—1200 has no Bronsted acid sites but has an amount of Lewis acid sites of 0.004 mmol/g. It is described that in a case in which Al₂O₃—1200 is used, the conversion rate is about 1.2%. Since the amount of Lewis acid sites in Al₂O₃—1200 is less than 0.005 mmol/g, it is presumed that the conversion rate is low due to a small number of active sites of the catalyst. Furthermore, Non-Patent Document 1 describes a method of producing trifluoroethylene using Al₂O₃—1150, which contains 64.3% by mass of α-alumina and has an amount of acid sites of 0.006 mmol/g. It is also described that in a case in which Al₂O₃—1150 is used, the conversion rate is about 1.2%.

It should be noted that, according to Non-Patent Document 1, when a supply amount of the fluorocarbon to the catalyst is calculated, it is 307 kg/h/m³, and since the raw material feed rate is set at a low level, the conditions allow for a high conversion rate to be exhibited. However, when the raw material feed rate is low, industrial productivity decreases, and therefore it is required to increase the raw material feed rate. In Non-Patent Document 1, in a case in which the raw material feed rate is increased, the conversion rate becomes lower than the above-mentioned value.

Furthermore, Non-Patent Document I describes a method of producing trifluoroethylene using alumina catalysts having an amount of acid sites of from 0.065 to 0.362 mmol/g. These alumina catalysts are composed of γ-alumina, θ-alumina and the like, and do not contain α-alumina. When these alumina catalysts are used, although the initial conversion rate is high, the conversion rate after 15 hours significantly decreases. This is presumed to be because, as described above, Y-alumina, θ-alumina and the like, which readily undergo changes in crystal structure under high-temperature conditions, are used.

It should be noted that, in Non-Patent Document 1, production is carried out under conditions in which the initial conversion rate is about 28% or higher and the conversion rate after 10 hours is 20% or higher, which are relatively high conversion rates, and in this case, the conversion rate after 15 hours markedly decreases. This is considered to be because, when the initial conversion rate and the like are excessively high, the amount of generated hydrogen fluoride increases, thereby promoting fluorination of the catalyst, which is one of the factors causing catalyst deactivation. As the fluorination of the catalyst progresses, coking of the catalyst tends to proceed more readily, and as a result, the catalyst is more likely to deactivate. In Non-Patent Document 1, in a case in which the production time is further extended to 50 hours, it is assumed that catalyst deactivation will progress further and the conversion rate will decrease even more.

Accordingly, as in the method of producing a fluoroolefin of the second embodiment, by suppressing the conversion rate after 10 hours from the contact of the fluorocarbon with the catalyst to 7 to 13%, the conversion rate retention after 50 hours can be maintained at 69% or higher. As an effective measures for suppressing the conversion rate after 10 hours to 7 to 13%, it is preferable to use the catalyst according to the first embodiment.

### (Fluorocarbon Represented by Formula (1))

In the fluoroolefin production method in the present disclosure, a fluorocarbon represented by the following Formula (1) is used as a raw material.

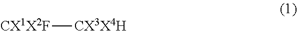

In Formula (1), X¹, X², X³ and X⁴ each independently represent a hydrogen atom or a fluorine atom, provided that at least one of X¹, X², X³ or X⁴ is a fluorine atom.

Examples of the fluorocarbon represented by Formula (1) include the following compounds.
CHF₂CH₃: 1,1-difluoroethane (HFC-152a)
CH₂FCH₂F: 1,2-difluoroethane (HFC-152)
CF₃CH₃: 1,1,1-trifluoroethane (HFC-143a)
CHF₂CH₂F: 1,1,2-trifluoroethane (HFC-143)
CF₃CH₂F: 1,1,1,2-tetrafluoroethane (HFC-134a)
CHF₂CHF₂: 1,1,2,2-Tetrafluoroethane (HFC-134)
CF₃CHF₂: 1,1,1,2,2-Pentafluoroethane (HFC-125)

Among these, the fluorocarbon represented by Formula (1) is preferably at least one selected from the group consisting of HFC-143a, HFC-143, HFC-134a, and HFC-134, from the viewpoint of reducing side reactions and suppressing the production of by-products. Furthermore, the fluorocarbon represented by Formula (1) is preferably HFC-134a, since one kind of fluoroolefin can be obtained with high selectivity.

### (Fluoroolefin Represented by Formula (2))

The fluoroolefin production method in the present disclosure produces a fluoroolefin represented by the following Formula (2) as the reaction product.

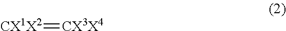

In Formula (2), X¹, X², X³ and X⁴ each independently represent a hydrogen atom or a fluorine atom, provided that at least one of X¹, X², X³ or X⁴ is a fluorine atom.

Examples of the Fluoroolefin represented by Formula (2) include the following compounds:
CHF═CH₂: Fluoroethylene
CF₂=CH₂: 1,1-Difluoroethylene (HFO-1132a)
CHF═CHF: 1,2-Difluoroethylene (HFO-1132 (E), HFO-1132 (Z))
CHF═CF₂: Trifluoroethylene (HFO-1123)
CF₂=CF₂: Tetrafluoroethylene

Among these, from the viewpoint of usefulness as a refrigerant composition, the fluoroolefin represented by Formula (2) is preferably at least one selected from the group consisting of HFO-1132, HFO-1132a, and HFO-1123.

In particular, in the fluoroolefin production method in the present disclosure, from the viewpoint of allowing the reaction to proceed more selectively, it is preferable that the fluorocarbon is HFC-134a and the fluoroolefin is HFO-1123.

In addition to the fluoroolefin represented by Formula (2), it is also possible to produce an olefin other than the fluoroolefin of Formula (2), and as the olefin other than Formula (2), ethylene may be included.

### (Catalyst)

In the fluoroolefin production method in the present disclosure, the fluorocarbon represented by Formula (1) is contacted with a catalyst.

The catalyst contacted with the fluorocarbon contains 65% by mass or more of α-alumina and has an amount of Lewis acid sites of from 0.005 to 0.10 mmol/g as measured by ammonia temperature-programmed desorption method.

Alumina is a dehydrated product of aluminum hydroxide, and its properties vary depending on the degree of dehydration and the degree of crystallinity. Alumina exists in various types such as γ-alumina, θ-alumina, and α-alumina, depending on its crystalline structure, γ-alumina and 0-alumina are called active alumina, have a higher free energy of formation than α-alumina, and are thermodynamically unstable. The high-temperature stable phase with a high degree of crystallinity is α-alumina, which has a small specific surface area but is considered to be thermally stable and to have high thermal conductivity. The catalyst used in the fluoroolefin production method in the present disclosure contains α-alumina. Compared with other alumina structures, α-alumina has a higher barrier to conversion from Al—O to A-F in the presence of hydrogen fluoride, and by using a catalyst containing α-alumina, it is possible to suppress the formation of AlF₃. The formation of AlF₃ is considered to lead to catalyst deactivation, a decrease in selectivity, and the like.

The presence of α-alumina in the catalyst can be confirmed by the diffraction pattern obtained using X-ray diffraction method, in other words, XRD (X-ray diffractometer). Commercially available XRD equipment can be used, such as “Smart Lab” manufactured by Rigaku Corporation. The presence of peaks at d=26.62, 35.21, 37.85, 43.43, 52.65, and 57.61 Å in the diffraction pattern indicates the presence of α-alumina. This analysis is to be performed on the catalyst immediately before contact with fluorocarbon, or on the catalyst in which the same state as that immediately before being brought into contact with the fluorocarbon is reproduced.

By the catalyst containing 65% by mass or more of α-alumina the durability of the catalyst is high, and a decrease in conversion rate is suppressed even in long-term production.

The fact that a catalyst contains 65% by mass or more of α-alumina can be confirmed by Rietveld analysis of the crystal structure by XRD. Specifically, the peaks obtained by XRD measurement of the catalyst are compared with known peak models derived from respective alumina structures, and by performing Rietveld analysis, the mass ratio of each crystal structure is calculated.

The catalyst contains α-alumina at 65% by mass or more, preferably 70% by mass or more, more preferably 75% by mass or more, even more preferably 80% by mass or more, and particularly preferably 85% by mass or more, and may contain 100% by mass.

The catalyst may contain a compound other than α-alumina. Examples of the compound other than α-alumina include alumina with a crystal structure different from that of α-alumina and an oxide other than alumina. Examples of alumina with a crystal structure different from that of α-alumina include β-alumina, γ-alumina, θ-alumina, η-alumina, boehmite, and gibbsite. Examples of the oxide other than alumina include chromium oxide, copper oxide, iron oxide, nickel oxide, magnesium oxide, zinc oxide, and zirconium oxide.

The catalyst may also contain a compound other than α-alumina, such as aluminum oxide fluoride and aluminum fluoride, which are fluorinated versions of α-alumina.

α-Alumina may function not only as a catalyst but may also as a support while functioning as a catalyst. Furthermore, α-alumina may be supported on a support other than α-alumina.

Examples of the support include carbon, β-alumina, γ-alumina, zirconia, silica, and titania.

The catalyst has an amount of Lewis acid sites of from 0.005 to 0.10 mmol/g as measured by ammonia temperature-programmed desorption method, preferably from 0.005 to 0.05 mmol/g, and more preferably from 0.008 to 0.05 mmol/g from the viewpoint of further suppressing a decrease in conversion rate after 100 hours.

The catalyst may be one that has undergone a drying treatment, one that has undergone an activation treatment, one that has undergone both, or one that has undergone neither. A state of the catalyst in the present disclosure is assumed to be a state of the catalyst at the start of the reaction (immediately before the start of the reaction). In a case in which each treatment is performed, the catalyst after the treatment is a subject for measurement of Lewis acid sites. In a case in which an amount of acid sites does not substantially change even after various treatments, Lewis acid sites may be measured on the catalyst before treatment.

The ammonia temperature-programmed desorption method involves adsorbing ammonia (NH₃) onto a test sample to be measured, continuously raising the temperature at a constant heating rate, and measuring an amount of ammonia desorbed and the desorption temperature. An amount of acid in the test sample to be measured can be determined from the amount of NH₃ desorbed. Furthermore, because NH₃ adsorbed on weak acid sites desorbs at low temperatures and NH₃ adsorbed on strong acid sites desorbs at high temperatures, an acid strength of the test sample to be measured can also be measured.

In the present disclosure, a peak intensity of an amount of ammonia desorbed at temperatures below 200° C. is taken as an amount of Bronsted acid sites, and a peak intensity of an amount of ammonia desorbed at temperatures above 200° C. is taken as an amount of Lewis acid sites, thereby distinguishing between Bronsted acid sites and Lewis acid sites.

Note that Lewis acid sites are an acid site that accepts an electron pair from a partner molecule, while a Bronsted acid site is an acid site that donates a proton to a partner molecule.

Ammonia temperature-programmed desorption method (NH₃-TPD) measurements are performed using a catalyst analyzer (for example, “BELCAT II” manufactured by MicrotracBEL). In measurement, a temperature is raised to 810° C. at a rate of 10° C./min and held for 10 minutes. Helium is used as a carrier gas at a flow rate of 50 mL/min (sccm).

Prior to measurement, the catalyst may be pretreated. Examples of pretreatment include performing processes 1 through 6 below in order. The temperature is then raised as described above to desorb NH₃ from the catalyst. The desorption temperature and desorption amount are measured, and an amount of Lewis acid sites is analyzed. The NH₃ used in process 5 below is diluted with He, resulting in an NH₃ concentration of 5.05% by volume. Therefore, a gas composition of the following process 5 is He: 90+10×0.9495=99.495 sccm, NH₃: 10×0.0505=0.505 sccm.

TABLE 1

| | | Flow Rate | Time | Set temperature |
|---|---|---|---|---|
| | Gas | (sccm) | (min) | (° C.) |
| 1 | He | 50 | 30 | 400 |
| 2 | He | 50 | 60 | 400 |
| 3 | He | 50 | 1 | 50 |
| 4 | He | 50 | 15 | 50 |
| 5 | He and NH₃ | He: 90, NH₃: 10 | 30 | 50 |
| 6 | He | 50 | 15 | 50 |

A catalyst form is not particularly limited and may be powder, pellet, or spherical.

α-alumina is preferably in a molded body such as spheres or pellets, from the viewpoint of excellent filling properties when being filled in a reactor, excellent flowability of reaction gas, and ease of handling in used for around 10 hours.

The molded body differs from a powder and is obtained, for example, by placing powder in a mold and compression-molding it.

### (Reaction Conditions)

In the fluoroolefin production method in the present disclosure, the feed gas need only contain the fluorocarbon represented by Formula (1), and may also contain a component other than the fluorocarbon represented by Formula (1). The feed gas may consist solely of the fluorocarbon represented by Formula (1), or may contain isomers, disproportionation products, impurities, and the like obtained during the production of the fluorocarbon represented by Formula (1). From the viewpoint of suppressing side reactions and catalyst deactivation, the feed gas preferably contains, in addition to the fluorocarbon represented by Formula (1), an inert gas such as nitrogen, argon, helium, carbon dioxide, or octafluorocyclobutane. By an inert gas, the desired product and hydrogen fluoride, which is a by-product, can be diluted. A content of the fluorocarbon represented by Formula (1) with respect to a total amount of the feed gas is preferably 60% by mol or more, more preferably 70% by mol or more, even more preferably 75% by mol or more, and particularly preferably 80% by mol or more.

The fluoroolefin production method in the present disclosure may be carried out in the gas phase or may be carried out in the liquid phase. Because the fluorocarbon represented by Formula (1) is a gas at room temperature, it is preferable to contact the fluorocarbon with the catalyst in the gas phase.

As the reactor for bringing the fluorocarbon into contact with the catalyst, any reactor may be used as long as it can withstand the temperature and pressure described later, and a shape and structure are not particularly limited. Examples of the reactor include a cylindrical vertical reactor. Examples of a material of the reactor include glass, stainless steel, iron, nickel, and alloys primarily composed of iron or nickel. The reactor may be equipped with a heating means, such as an electric heater, for heating the interior of the reactor.

The catalyst may be accommodated in any type of fixed bed, fluidized bed, or moving bed. In the case of a fixed bed, it may be either a horizontal or vertical fixed bed.

The reaction type may be either a flow type or a batch type.

In the fixed bed reactor, various molded bodies of catalyst-supported carriers are filled in order to reduce the pressure loss of the reaction fluid. Similarly to the fixed-bed reactor, a system in which the catalyst is filled, moved by its gravity, and withdrawn from the bottom of the reactor for regeneration is called a moving bed. In a fluidized-bed reactor, in order to operate so that the catalyst layer exhibits characteristics as if it were a fluid by the reaction fluid, the catalyst particles are suspended in the reaction fluid and move within the reactor. A fixed-bed reactor is preferable in that the options for the shape of the catalyst are wide and catalyst wear can be suppressed. As the fixed-bed reactor, there are a tubular reactor and a tank-type reactor, and the tubular reactor is preferable because of ease in controlling the reaction temperature. Furthermore, a multitubular heat-exchange type reactor in which a large number of reaction tubes with a small tube diameter are arranged in parallel and a heat medium is circulated on the outside can be employed. In a case in which a plurality of reactors are provided in series, a plurality of catalyst layers are thereby provided. The catalyst layer may have at least one stage, and may have two or more stages.

From the viewpoint of improving conversion rate, the fluoroolefin production method in the present disclosure is preferably carried out using a flow-through system using a fixed-bed reactor (particularly a vertical fixed-bed reactor).

From the viewpoint of improving industrial productivity, an amount of fluorocarbon supplied to the catalyst (raw material feed rate) is preferably 350 kg/hr/m³ or more, more preferably 375 kg/hr/m³ or more, and even more preferably 400 kg/hr/m³ or more. Furthermore, from the viewpoint of maintaining a constant or higher conversion rate, the raw material feed rate is preferably 3,000 kg/hr/m³ or less, more preferably 2,500 kg/hr/m³ or less, and even more preferably 2,000 kg/hr/m³ or less.

The raw material feed rate refers to an amount of fluorocarbon (raw material) supplied per 1 m³ of catalyst per unit time (kg/hr). It is preferable that an amount of fluorocarbon brought into contact with the catalyst by being supplied be within the above range.

In the fluoroolefin production method in the present disclosure, it is preferable to bring the fluorocarbon into contact with the catalyst at a temperature of from 300 to 800° C., more preferably at a temperature of from 400 to 700° C. and even more preferably at a temperature of from 400 to 600° C. In a case in which the contact temperature is 300° C. or higher, the conversion rate of the fluoroolefin is improved. On the other hand, in a case in which the contact temperature is 800° C. or lower, the decomposition of the fluoroolefin can be suppressed.

In a case in which the contact temperature is 300° C. or higher, the reaction proceeds appropriately. On the other hand, in a case in which the contact temperature is 800° C. or lower, a decrease in selectivity due to cleavage of carbon-carbon bonds of the raw material and a disproportionation reaction of the product (unsaturated compound) are suppressed.

Since the dehydrofluorination reaction is generally an endothermic reaction, maintaining an appropriate reaction temperature can prevent a decrease in conversion rate. As the reaction temperature in the catalyst layer increases, the conversion rate of the raw material increases. Therefore, it is preferable to maintain the reaction temperature in the catalyst layer at a desired temperature to maintain a high conversion rate. One way to maintain the reaction temperature in the catalyst layer at a desired temperature is to externally heat the catalyst layer using a heat transfer medium or the like. Catalysts typically deteriorate over time as the reaction progresses. Even when a decrease in the conversion rate of the raw material occurs due to catalyst deterioration, the decrease in conversion rate can be prevented by heating the catalyst layer with a heat transfer medium or the like and appropriately maintaining or increasing the reaction temperature. When maintaining or increasing the temperature of the catalyst layer, it is preferable to limit the temperature increase to 50° C. or less to prevent rapid catalyst degradation.

At the start of the reaction, the reaction zone begins at the feed gas inlet. As the catalyst at the feed gas inlet deteriorates over time as the reaction progresses, the reaction zone moves downstream in the gas flow direction. Since the low-temperature product gas generated at the reaction zone flows into a downstream vicinity area of the reaction zone, this downstream vicinity area is usually at the lowest temperature within the catalyst layer. In the present disclosure, the temperature of this lowest-temperature region of the catalyst layer is referred to as the “lowest temperature of the catalyst layer.” The temperature further downstream from the downstream vicinity area usually becomes higher than the lowest temperature of the catalyst layer as it moves away from the reaction zone.

In the fluoroolefin production method in the present disclosure, the feed gas containing a fluorocarbon may be supplied to the reactor at room temperature. However, it is preferable to appropriately heat (preheat) the feed gas before supplying it to the reactor. In a case in which preheating is performed, the feed gas is preferably heated to a temperature of from 80 to 600° C. before being supplied to the reactor. In a case in which preheating is performed at 80° C. or higher, the internal temperature of the reactor is less likely to decrease, and the set conversion rate can be more easily achieved. In a case in which preheating is performed at 600° C. or lower, the internal temperature of the reactor is less likely to increase, undesirable reactions are suppressed, and the selectivity is improved.

The dehydrofluorination reaction in the present disclosure is a reaction in which molecules increase, and therefore, increasing the pressure makes the forward reaction unfavorable.

The pressure when contacting the fluorocarbon with the catalyst is not particularly limited, but from the viewpoint of improving conversion rate, from-0.05 to 2 MPa is preferred, from-0.01 to 1 MPa is more preferred, and from atmospheric pressure to 0.5 MPa is even more preferred.

In the present disclosure, pressure refers to gauge pressure.

A contact time (seconds) between the fluorocarbon and the catalyst is preferably from 0.5 to 100.0 seconds, more preferably from 1.0 to 50.0 seconds, and even more preferably from 2.0 to 20.0 seconds.

The above contact time (seconds) is calculated using the following Formula:

$$Contact time ( seconds ) = [ length of catalyst filled in a reactor ( cm ) ] / [ linear velocity ( cm / sec ) ]$$

Linear velocity refers to a speed at which the fluorocarbon passes through the catalyst per unit time.

Furthermore, a contact time (g·sec/mL) between the fluorocarbon and the catalyst is preferably from 1 to 200 g·sec/mL, more preferably from 5 to 175 g·sec/mL, even more preferably from 7 to 150 g·sec/mL, and particularly preferably from 10 to 125 g·sec/mL. In a case in which the contact time (g·sec/mL) is 1 g·sec/mL or more, the conversion rate can be improved. In a case in which the contact time (g·sec/mL) is 200 g, sec/mL or less, equipment cost can be reduced.

The contact time (g·sec/mL) is calculated using the following Formula:

$$Contact time ( g \cdot sec / mL ) = [ Amount of Catalyst filled ( g ) ] / [ Fluorocarbon flow rate ( mL / sec ) ]$$

To further reduce the decrease in conversion rate, it is preferable to contact the fluorocarbon with the catalyst in the presence of an inert gas. The inert gas is preferably at least one selected from the group consisting of nitrogen, helium, argon, octafluorocyclobutane, and carbon dioxide. Among them, nitrogen is preferred as the inert gas. In the present disclosure, an inert gas refers to a gas that is inert to the raw material.

A molar ratio of the fluorocarbon to inert gas in the gas phase is preferably from 0.1 to 30, more preferably from 0.5 to 25.

From the viewpoint of further suppressing a decrease in conversion rate, it is preferable that the fluorocarbon and catalyst be contacted in the gas phase in the presence of water, and that a concentration of the water be less than 500 ppm by mass with respect to a total amount of the feed gas containing a fluorocarbon.

In a case in which a dehydrofluorination reaction is carried out in the gas phase in the presence of water, water adsorbs to the Lewis acid sites on the alumina-containing catalyst surface. It is presumed that, by setting the concentration of water to less than 500 mass ppm with respect to the total amount of the raw material gas containing the fluorocarbon, collapse of Lewis acid sites on the surface of the alumina-containing catalyst, which leads to the formation of structures similar to Bronsted acid sites, is suppressed, and a decrease in the activity of the catalyst is prevented, thereby suppressing a decrease in catalyst activity. It is presumed that, as a result, the conversion rate becomes higher and the desired product can be obtained with high selectivity. The concentration of the water is preferably 300 ppm by mass or less, more preferably 100 ppm by mass or less, even more preferably 50 ppm by mass or less, and particularly preferably 10 ppm by mass or less, in order to further improve the conversion rate and obtain the desired compound with even higher selectivity. Although a lower water concentration is preferable, from the viewpoint that the cost of dehydration treatment of the fluorocarbon and the inert gas and process control become difficult, it is preferably 0.5 ppm by mass or more, and more preferably 1 ppm by mass or more.

Examples of a common method for measuring the concentration of water include using a commercially available Karl Fischer moisture content meter.

The concentration of the water refers to the water content of the feed gas when the fluorocarbon is brought into contact with the catalyst. The concentration of the water may also be substituted with the water content of the feed gas before it is introduced into the reactor.

The fluoroolefin production method in the present disclosure preferably further includes drying the catalyst before contacting the fluorocarbon with the catalyst. Drying the catalyst removes water from the catalyst, increasing its reactivity with fluorocarbons and improving the conversion rate.

A method for drying the catalyst is not particularly limited. The catalyst may be dried before being filled into the reactor or may be dried after being filled into the reactor. Drying the catalyst after filling the reactor is preferred because the reactor can also be preheated together with drying of the catalyst. Specifically, it is preferable to fill the catalyst into the reactor and heat the reactor while flowing an inert gas through it to dry the catalyst.

In the fluoroolefin production method in the present disclosure, hydrogen fluoride is produced as a by-product. Hydrogen fluoride fluorinates oxides contained in the catalyst, strengthening its acidity. Therefore, it is preferable to reduce the hydrogen fluoride concentration. In a case in which the hydrogen fluoride concentration is reduced, the selectivity is maintained. deactivation of the catalyst is suppressed, and a decrease in reaction activity due to a decrease in a specific surface area of the catalyst is suppressed.

One method for reducing the hydrogen fluoride concentration is to dilute it with an inert gas. Since the use of an inert gas increases the energy load of the purification process after the reaction, it is preferable that the use of the inert gas be appropriately controlled.

It is preferable that the hydrogen fluoride concentration during the reaction be 15% by mol or less. From the viewpoint of extending the catalyst life, the hydrogen fluoride concentration is more preferably 13% by mol or less, even more preferably 10% by mol or less, particularly preferably 8% by mol or less, and most preferably 7% by mol or less. Furthermore, from the viewpoint of productivity and the energy load of the purification process, the hydrogen fluoride concentration is preferably 0.5% by mol or more, more preferably 0.8% by mol or more, even more preferably 1.0% by mol or more, particularly preferably 1.3% by mol or more, and most preferably 1.5% by mol or more.

The fluoroolefin production method in the present disclosure is preferably carried out in the presence of an oxidizing agent. From the viewpoint that the conversion rate is high and the desired compound can be obtained with high selectivity, the oxidizing agent is preferably oxygen, chlorine, bromine, or iodine. Among them, oxygen is more preferred as the oxidizing agent. In the present disclosure, a concentration of the oxidizing agent is preferably from 0.01 to 21% by mol with respect to the feed gas. The concentration of the oxidizing agent is more preferably from 1 to 20% by mol, even more preferably from 5 to 18% by mol, and particularly preferably from 7.5 to 16% by mol, with respect to the raw material compound, from the viewpoint of further improving the conversion rate and obtaining the desired compound with even higher selectivity.

In the present disclosure, the conversion rate refers to a ratio (mol %) of a total molar amount of compounds other than the raw material compound contained in the effluent gas from the reactor outlet to a molar amount of the raw material compound supplied to the reactor.

In general, a higher conversion rate is preferable from the viewpoint of productivity. However, in the dehydrofluorination reaction in the present disclosure, it is preferable to appropriately control the conversion rate. Controlling the conversion rate reduces the concentration of hydrogen fluoride produced in the gas phase, which is thought to suppress catalyst deactivation by hydrogen fluoride. While diluting the gas phase with an inert gas is one possible method, using an inert gas increases the energy load in the purification process after the reaction, and therefore, using excessive inert gas is not preferable. From the viewpoint of extending the catalyst life, it is preferable to keep the hydrogen fluoride concentration during the reaction to 15% by mol or less.

In light of the above, it is preferable to select operating conditions such that a conversion rate 10 hours after contacting the fluorocarbon with the catalyst is from 7 to 13%, and it is more preferable to select operating conditions such that the conversion rate after 10 hours is from 7.5 to 12.5%.

It is preferable to select operating conditions such that a conversion rate 50 hours after contacting the fluorocarbon with the catalyst is 4.0% or higher, and more preferable 4.5% or higher, even more preferably 5.0% or higher, and most preferably 5.5% or higher.

It is preferable to select operating conditions such that a conversion rate 100 hours after contacting the fluorocarbon with the catalyst is 1.0% or higher, more preferably 2.0% or higher, even more preferably 3.0% or higher, and most preferably 4.0% or higher.

A conversion retention rate, which is a ratio of the conversion rate after 50 hours to the conversion rate after 10 hours, is preferably 69% or higher. Furthermore, a conversion retention rate after 100 hours from contacting the fluorocarbon with the catalyst is preferably 30% or higher, more preferably 40% or higher, and even more preferably 50% or higher.

In particular, in the fluoroolefin production method in the present disclosure, by using HFC-134a as the fluorocarbon, the conversion rate after 10 hours does not become too high and catalyst deactivation is suppressed.

Furthermore, by using a catalyst containing 65% by mass or more of α-alumina and having an amount of Lewis acid sites of from 0.005 to 0.10 mmol/g as measured by ammonia temperature-programmed desorption method, the conversion rate after 10 hours does not become too high and catalyst deactivation is suppressed.

In the present disclosure, selectivity refers to a ratio (mol %) of a molar amount of the desired product contained in the reactor outlet gas to a total molar amount of compounds other than the raw material compound contained in the reactor outlet gas.

A selectivity of 100% is preferable because it eliminates the need for a post-reaction purification process, but side reactions may occur within the reaction temperature range required to achieve a desired conversion rate. A higher selectivity is preferable because it reduces an amount of waste, the energy load of the post-reaction purification process, and extends the catalyst life.

A selectivity 50 hours after contacting the fluorocarbon with the catalyst is preferably 90% or higher, more preferably 93% or higher, and even more preferably 95% or higher.

In particular, in the fluoroolefin production method in the present disclosure, HFO-1123 can be obtained with high selectivity by using HFC-134a as the fluorocarbon.

Examples of a compound other than the raw material compound and the desired product contained in the reactor outlet gas include hydrogen fluoride, carbon monoxide, carbon dioxide, and water. For example, in a case in which HFC-134a is used as the fluorocarbon raw material compound, the other compound may include HFC-134, 1,1-difluoroethylene (VdF), E/Z-1,2-difluoroethylene (HFO-1132 (E)/(Z)), and the like.

### EXAMPLES

Hereinafter, the present disclosure will be described more specifically by Examples; however, the present disclosure is not limited to the following Examples as long as the gist thereof is not exceeded.

Examples 1 to 6 are examples, and Examples 7 to 8 are comparative examples.

### Example 1

α-alumina (product name: “α-alumina, average particle size 0.5 μm.” manufactured by FUJIFILM Wako Pure Chemical Corporation) was weighed in an amount of 1 mL and used as a catalyst. A stainless steel (SUS304) reactor tube with an inner diameter of 1.02 cm and a length of 30 cm was filled with the catalyst and placed in a tubular electric furnace. The catalyst-filled section was heated to 475° C. in the furnace while nitrogen was circulating, dehydrating the catalyst. A nitrogen/HFC-134a (0.1/1 mol/mol) mixed gas was then passed through the tube for a contact time of 4.7 seconds to carry out the dehydrogenation reaction to HFO-1123.

A water concentration in the nitrogen/HFC-134a (0.1/1 mol/mol) mixed gas was measured using a Karl Fischer moisture content analyzer and found to be 5 ppm by mass.

### Examples 2 to 8

In Examples 2 to 8, except that the catalyst was changed and the various conditions were changed to the values shown in Table 2, the dehydrofluorination reaction was carried out in the same manner as in Example I.

Hereinafter, the catalysts used in Examples 2 to 8 will be described.

### Example 2

α-alumina (product name “SA52124”, manufactured by Saint-Gobain) was used as the catalyst.

### Example 3

α-alumina (product name “SA52238”, manufactured by Saint-Gobain) was used as the catalyst.

### Example 4

α-alumina (product name “FGL-40”, manufactured by Iwatani Chemical Industries, Ltd.) was used as the catalyst.

### Example 5

α-alumina (product name “LT303D”, manufactured by Nippon Light Metal Co., Ltd.) was used as the catalyst.

### Example 6

α-alumina (product name “C500”, manufactured by Nippon Light Metal Co., Ltd.) was used as the catalyst.

### Example 7

γ-alumina (product name “N612N”, manufactured by JGC Catalysts and Chemicals Ltd) was used as the catalyst.

### Example 8

A catalyst consisting primarily of 0-alumina with a portion of α-alumina (product name “SA3177”, manufactured by Saint-Gobain) was used.

An amount of the Lewis acid sites of each catalyst was determined by NH₃-TPD using a “BELCAT II” manufactured by a Microtrac BEL. The measurement conditions were as described above. An amount of the Lewis acid sites measurement was performed before the catalyst was filled into a reaction tube.

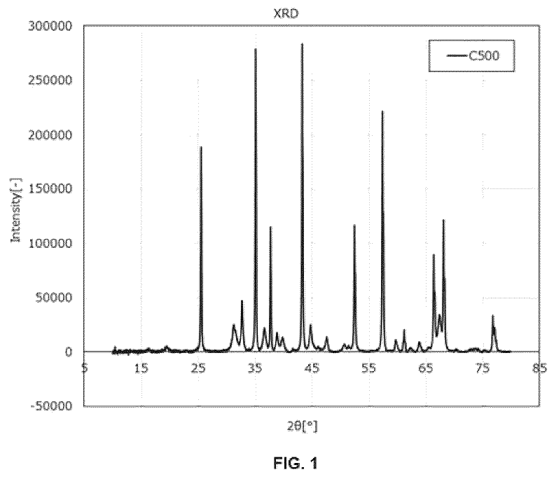

XRD measurement was performed on the catalyst of Example 6 using a X-ray diffractometer “SmartLab” manufactured by Rigaku Corporation. The resulting diffraction pattern is shown in FIG. 1. The diffraction pattern shown in FIG. 1 contains peaks at 2θ=26.62, 35.21, 37.85, 43.43, 52.65, and 57.61 Å, confirming the presence of α-alumina.

Furthermore, the presence of peaks at 2θ=31.51, 32.78, 36.74, 38.87, 44.86, and 67.40 A confirms the presence of θ-alumina.

Rietveld analysis was then performed on the diffraction peaks of each crystalline phase observed above, confirming that the α-alumina content in the catalyst was 65.2% by mass and the 0-alumina content was 34.8% by mass.

In the same manner, the content of α-alumina was determined by XRD measurement also for the catalysts of Examples 1 to 5 and 7 to 8.

A contact time (seconds) was calculated using the following Formula:

$$Contact time ( seconds ) = [ length of catalyst filled in a reactor ( cm ) ] / [ linear velocity ( cm / sec ) ]$$

Linear velocity refers to a speed at which the fluorocarbon passes through the catalyst per unit time.

A contact time (g·sec/mL) was calculated using the following Formula:

$$Contact time ( g sec / mL ) = [ Amount of Catalyst filled ( g ) ] / [ Fluorocarbon flow rate ( mL / sec ) ]$$

In the reactions of Examples 1 to 8, the product gas (hereinafter also referred to as “reactor outlet gas”) extracted from the reactor outlet 10 and 50 hours after the start of the reaction was analyzed using a gas chromatograph. Specifically, a gas chromatograph (product name “GC6850” manufactured by Agilent) was equipped with a column (product name “DB-1301” manufactured by Agilent, length 60 m, inner diameter 0.25 mm, film thickness 1 μm) for analysis. A molar amount calculated from an area ratio (GCArea %) of the reactor outlet gas was used to calculate the conversion rate of HFC-134a and the selectivity of HFO-1123.

### (Conversion Rate of HFC-134a)

This refers to a ratio (mol %) of a total molar amount (MI) of components other than HFC-134a contained in the reactor outlet gas to a total molar amount (M₁₃₄ₐ) of HFC-134a fed to the reactor.

$$Conversion Rate of HFC - 134 a ( \% ) = ( M 1 / {M}_{134 a} ) \times 100$$

### (Selectivity of HFO-1123)

This refers to a ratio (mol %) of a molar amount (M1123) of HFO-1123 contained in the reactor outlet gas to the total molar amount (MI) of compounds other than HFC-134a contained in the reactor outlet gas.

$$Selectivity of HFO - 1123 ( \% ) = ( {M}_{1123} / M 1 ) \times 100$$

TABLE 2

| | | Example 1 | Example 2 | Example 3 | Example 4 | Example 5 | Example 6 | Example 7 | Example 8 |
|---|---|---|---|---|---|---|---|---|---|
| Catalyst | α-alumina | 100 | 100 | 100 | 100 | 100 | 65.2 | 0 | <50 |
| (% by mass) | θ-alumina | 0 | 0 | 0 | 0 | 0 | 34.8 | 0 | ≥50 |
| | γ-alumina | 0 | 0 | 0 | 0 | 0 | 0 | 100 | 0 |
| | Amount of | 0.0055 | 0.0054 | 0.019 | 0.0095 | 0.0067 | 0.010 | 0.12 | 0.11 |
| | Lewis acid | | | | | | | | |
| | sites [mmol/g] | | | | | | | | |
| Contact Temperature [° C.] | | 450 | 450 | 450 | 450 | 450 | 450 | 450 | 450 |
| Pressure [MPaG] | | 0 | 0 | 0 | 0 | 0 | 0 | 0 | 0 |
| Molar Ratio of Fluorocarbon/N₂ | | 10 | 10 | 10 | 10 | 10 | 10 | 10 | 10 |
| [mol/mol] | | | | | | | | | |
| Contact Time [sec] | | 4.7 | 4.7 | 4.7 | 4.7 | 4.7 | 4.7 | 4.7 | 4.5 |
| Contact Time [g · sec/mL] | | 14 | 15 | 11 | 19 | 21 | 3.9 | 13 | 13 |
| Raw Material Feed Rate | | 1257 | 1257 | 1257 | 1257 | 1257 | 1257 | 1257 | 1257 |
| [kg/hr/m³] | | | | | | | | | |
| Conversion | After 10 hr | 10 | 8.4 | 10.8 | 8.3 | 12.0 | 11.9 | 6.9 | 10.1 |
| Rate [%] | After 50 hr | 8.1 | 9.2 | 7.6 | 5.8 | 10.3 | 10.1 | 4.0 | 6.9 |

| | | | | | | | | |
|---|---|---|---|---|---|---|---|---|
| Selectivity [%] | 99 | 99 | 99 | 98 | 99 | 99 | 97 | 99 |
| Conversion Rate Retention | 81 | 110 | 70 | 70 | 86 | 85 | 58 | 68 |
| Rate, after 50 hours [%] | | | | | | | | |

As shown in Table 2, Examples 1 to 6 include contacting a fluorocarbon represented by the following Formula (1) with a catalyst to produce a fluoroolefin represented by the following Formula (2), in which the catalyst comprises 65% by mass or more of α-alumina and has an amount of Lewis acid sites of from 0.005 to 0.10 mmol/g as measured by ammonia temperature-programmed desorption method. This resulted in a higher conversion rate than conventional methods, and suppressed a decrease in conversion rate during long-term production.

On the other hand, Example 7, which did not contain α-alumina and had an amount of Lewis acid sites exceeding 0.10 mmol/g, had an extremely low conversion rate compared to Examples 1 to 6.

Furthermore, Example 8, which contained α-alumina but had an amount of Lewis acid sites exceeding 0.10 mmol/g, had an equivalent conversion rate after 10 hours compared to Examples 1 to 6, but showed a significant decrease in conversion rate during long-term production such as 50 hours.

Furthermore, as shown by the results of Examples 1 to 6, Since this method includes a fluorocarbon represented by the following Formula (1) with a catalyst to produce a fluoroolefin represented by the following Formula (2), in which a conversion rate 10 hours after contacting the fluorocarbon with the catalyst is from 7.0 to 13.0%, it was found that a decrease in conversion rate was suppressed during long-term production.

### Examples 9 and 10

Furthermore, for the catalysts used in Examples 4 and 5, the dehydrofluorination reaction was carried out in the same manner as in Example 1, except that the various conditions were changed to the values listed in Table 3, and the results were designated as Examples 9 and 10. The conversion rate of HFC-134a and selectivity of HFO-1123 were calculated 10 hours and 100 hours after the start of the reaction.

TABLE 3

| | | Example 9 | Example 10 |
|---|---|---|---|
| Catalyst | α -alumina | 100 | 100 |
| (% by mass) | θ -alumina | 0 | 0 |
| | γ -alumina | 0 | 0 |
| | Amount of Lewis | 0.0095 | 0.0067 |
| | acid sites | | |
| | [mmol/g] | | |
| Contact Temperature [° C.] | | 450 | 450 |
| Pressure [MPaG] | | 0 | 0 |
| Molar Ratio of Fluorocarbon/N₂ | | 4 | 4 |
| [mol/mol] | | | |
| Contact Time [sec] | | 3.9 | 3.9 |
| Contact Time [g · sec/mL] | | 17 | 23 |
| Raw Material Feed Rate [kg/hr/m³] | | 1257 | 1257 |
| Conversion | After 10 hr | 8.3 | 5.9 |
| Rate [%] | After 100 hr | 4.8 | 1.4 |
| Selectivity [%] | | 98 | 97 |
| Conversion Rate Retention | | 58 | 24 |
| Rate, after 100 hours [%] | | | |

As shown in Table 3, Example 9, in which the amount of Lewis acid sites was within the range of from 0.008 to 0.05 mmol/g, was found to more effectively suppress a decrease in conversion rate during long-term production such as 100 hours, than Example 10, which was outside this range.

Note that the raw material feed rate in the examples was 1,257 kg/hr/m³, which is about four times larger than the raw material feed rate of 307 kg/h/m³ in Non-Patent Document 1, and is a condition that results in a lower conversion rate. However, even when the raw material feed rate is increased in this manner, it was found that when including contacting a fluorocarbon represented by the following Formula (1) with a catalyst to produce a fluoroolefin represented by the following Formula (2), in which the catalyst comprises 65% by mass or more of α-alumina and has an amount of Lewis acid sites of from 0.005 to 0.10 mmol/g as measured by ammonia temperature-programmed desorption method, the conversion rate is excellent and a decrease in conversion rate during long-term production is suppressed.

The disclosure of Japanese Patent Application No. 2023—072720 is incorporated herein by reference in its entirety. All literature, patent applications, and technical standards described herein are incorporated herein by reference to the same extent as if each individual literature, patent application, and technical standard were specifically and individually indicated to be incorporated by reference.

1. A method of producing a fluoroolefin, the method comprising contacting a fluorocarbon represented by the following Formula (1) with a catalyst to produce a fluoroolefin represented by the following Formula (2),
wherein the catalyst comprises 65% by mass or more of α-alumina and has an amount of Lewis acid sites of from 0.005 to 0.10 mmol/g as measured by ammonia temperature-programmed desorption method, 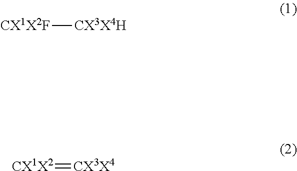
wherein, in Formulas (1) and (2), X¹, X², X³ and X⁴ each independently represent a hydrogen atom or a fluorine atom, provided that at least one of X¹, X², X³ or X⁴ is a fluorine atom.

2. The method of producing a fluoroolefin according to claim 1, wherein the amount of Lewis acid sites is from 0.005 to 0.05 mmol/g.

3. The method of producing a fluoroolefin according to claim 1, wherein the amount of Lewis acid sites is from 0.008 to 0.05 mmol/g.

4. The method of producing a fluoroolefin according to claim 1, wherein an amount of fluorocarbon supplied to the catalyst is 350 kg/hr/m³ or more.

5. The method of producing a fluoroolefin according to claim 1, wherein the fluorocarbon is at least one selected from the group consisting of 1,1,1-trifluoroethane, 1,1,2-trifluoroethane, 1, 1,2,2-tetrafluoroethane, and 1,1,1,2-tetrafluoroethane.

6. The method of producing a fluoroolefin according to claim 1, wherein the fluoroolefin is at least one selected from the group consisting of 1,2-difluoroethylene, 1,1-difluoroethylene, and trifluoroethylene.

7. The method of producing a fluoroolefin according to claim 1, wherein the fluorocarbon is 1,1,1,2-tetrafluoroethane, and the fluoroolefin is trifluoroethylene.

8. The method of producing a fluoroolefin according to claim 1, wherein the fluorocarbon and the catalyst are contacted at a temperature of from 300 to 800° C.

9. The method of producing a fluoroolefin according to claim 1, wherein:
the fluorocarbon and the catalyst are contacted in the presence of an inert gas; and
the inert gas is at least one selected from the group consisting of nitrogen, helium, argon, octafluorocyclobutane, and carbon dioxide.

10. The method of producing a fluoroolefin according to claim 1, further comprising drying the catalyst before contacting the fluorocarbon with the catalyst.

11. The method of producing a fluoroolefin according to claim 1, wherein:
the fluorocarbon and the catalyst are contacted in a gas phase in the presence of water; and
a concentration of the water is less than 500 ppm by mass with respect to a total amount of a feed gas containing the fluorocarbon.

12. A method of producing a fluoroolefin, the method comprising contacting a fluorocarbon represented by the following Formula (1) with a catalyst to produce a fluoroolefin represented by the following Formula (2),
wherein a conversion rate 10 hours after contacting the fluorocarbon with the catalyst is from 7.0 to 13.0%, and a conversion retention rate, which is a ratio of the conversion rate 50 hours after contact to the conversion rate after 10 hours, is 69% or higher. 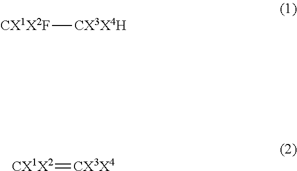
wherein, in Formulas (1) and (2), X¹, X², X³ and X⁴ each independently represent a hydrogen atom or a fluorine atom, provided that at least one of X¹, X², X³ or X⁴ is a fluorine atom.
